(19) United States
(12) Patent Application Publication
(10) Pub. No.: US 2022/0221409 A1
(43) Pub. Date: Jul. 14, 2022

(54) A SENSOR

(71) Applicant: IP2IPO Innovations Limited, London (GB)

(72) Inventors: Antoine Barbot, London (GB); Guang-Zhong Yang, London (GB); Dominic Wales, London (GB); Jang Ah Kim, London (GB); Salzitsa Yordanova Anastasova-Ivanova, London (GB); Burak Temelkuran, London (GB); Mohamed EMK Abdelaziz, London (GB)

(21) Appl. No.: 17/613,277

(22) PCT Filed: May 19, 2020
(86) PCT No.: PCT/GB2020/051217

§ 371 Date: Nov. 22, 2021

(30) Foreign Application Priority Data
May 23, 2019 (GB) 1907292.5

(51) Int. Cl. G01N 21/85; A61B 5/00; A61B 5/1473

(52) U.S. Cl. CPC G01N 21/8507; A61B 5/1473; A61B 5/0075; A61B 5/6852

(57) ABSTRACT

A sensor comprising an inlet and an outlet, a sensing chamber positioned between the inlet and the outlet, and a sensing element operatively connected to the sensing chamber, wherein the sensor comprises a first fibre formed from a drawable material, the fibre comprising a first channel extending between the inlet and the outlet, the sensing chamber being formed within the channel.

This invention relates to a sensor, and particularly, but not exclusively, to a sensor having combined electrical and optical sensors for use in in-vivo sensing.

A sensor of this type has particular application in the field of diagnostics. Sensors of this type are suitable for detecting diseases such as those identified below, although its to be understood that sensors of this type could be used in other applications.

Such a sensor has application in the respiratory system of a human or animal. Diseases such as pneumonia, both typical and atypical, lung cancers, chronic pulmonary disease (COPD, including emphysema and chronic bronchitis), cystic fibrosis, asthma, tuberculosis, bronchiectasis, sarcoidosis and other diseases may be diagnosed using sensors of this type.

In the urinary tract, urethral cancers, bladder cancers, ureter cancers, kidney cancers, pyelonephritis, urinary tract infection and other diseases may be diagnosed.

According to a first aspect of the invention there is provided a sensor comprising an inlet and an outlet, a sensing chamber positioned between the inlet and the outlet, and a sensing element operatively connected to the sensing chamber, wherein the sensor comprises a first fibre formed from a drawable material, the fibre comprising a first channel extending between the inlet and the outlet, the sensing chamber being formed within the channel.

By means of the present invention it is possible to carry out diagnostic tests in-vivo.

Because the sensor comprises a fibre, it may have very small dimensions, which facilitates insertion of the sensor into an appropriate part of the patient's body.

The sensor may have any convenient dimensions, and in some embodiments of the invention, the fibre forming the sensor is 0.3 m in length. In other embodiments of the invention, the fibre may be longer or shorter, and in some embodiments of the invention the fibre is approximately m in length.

The diameter of the fibre forming the sensor may be approximately 1 mm, and in some embodiments of the invention it may be less. In some embodiments of the invention the diameter of the fibre is 0.2 mm.

In addition, because the sensing chamber is formed within a channel which is itself formed within the first fibre, the sensing chamber may be protected from the environment in which the sensor is positioned. In particular, the sensing chamber may be protected from damaging frictional contact with the surrounding environment.

In embodiments of the invention, the sensor further comprises a seal, sealingly connected to the fibre. The seal may comprise, for example a wall of the first fibre, or may comprise a seal formed from a polymer such as UV cured glue.

The seal assists in protecting the sensing chamber from the environment.

The first channel may comprise a microfluidic flow channel. The microfluidic flow channel enables microfluidic connections to take place along the length of the fibre. The first channel may have any convenient or desirable dimensions, and in some embodiments of the invention, the first channel has a diameter within the range 0.05 mm to 0.5 mm.

In embodiments of the invention, the first channel comprises a microfluidic flow channel or groove.

In other words, the first channel comprises features which may be either inherently formed within the channel, or may be formed separate thereto, which features result, in microfluidic flow when a fluid passes through the channel.

In embodiments of the invention, the first channel comprises patterns etched into the wall of the first channel in the sensing chamber. These patterns will be referred to herein as microfluidic patterns. The microfluidic patterns cause microfluidic flow when a fluid is passed through the sensor.

The microfluidic patterns may have any desirable dimensions, and in some embodiments of the invention, the microfluidic patterns have dimensions of about 0.05 mm. In other embodiments of the invention microfluidic patterns may be smaller and larger, and in one embodiment of the invention microfluidic patterns have a dimension of 0.001 mm.

In embodiments of the invention, the sensor comprises a first pump operatively connected to the outlet of the sensor.

The first pump may be used in order to cause a fluid to flow through the sensor. A fluid may enter the sensor via the inlet and may then pass through the sensing chamber in order that fluid may be analysed by the sensing element which is operatively connected to the sensing chamber. By means of the first pump, the rate of flow of a liquid passing through the sensing element may be varied in order to suit the circumstances under which the sensor is being used.

In embodiments of the invention the first pump comprises a syringe pump.

In some embodiments of the invention the first pump comprises a reservoir pump operatively connected to a reservoir holding fluid.

In such embodiments of the invention, the pump is adapted to tune the pressure of the reservoir.

In some embodiments of the invention, there may be one or more syringe pumps and/or one or more reservoir pumps operatively connected to one or more reservoirs.

In embodiments of the invention comprising a syringe pump, the syringe pump may comprise a plunger operatively connected to a linear motor. The linear motor may be adapted to provide a constant speed to the plunger.

In such embodiments, there will be constant flow of fluid within the sensor. The flow may be either positive or negative depending on whether the plunger of the syringe is pushed or pulled by the linear motor. In other words, in such embodiments of the invention, the syringe pump may cause fluid to flow either into or out of the sensor.

In embodiments of the invention comprising a reservoir pump, the reservoir pump may be adapted to control pressure within a tuneable reservoir. If a positive pressure is applied by the reservoir pump, fluid contained in the reservoir will flow out of the reservoir. On the other hand, if a negative pressure is applied to the reservoir, fluid will flow into the reservoir.

In embodiments of the invention, the reservoir and the reservoir pump may be connected by means of a fluidic tube. There may be one or more such fluidic tubes. The fluidic tube(s) may be formed from polymer or glass, but other materials could also be used.

In embodiments of the invention, the one or more fluid tubes may be operatively connected to one or more channels extending between the inlet and the outlet of the sensor.

In embodiments of the invention, the sensing element comprises an optical sensor comprising a sensing optical fibre extending along the sensor such that at least a part of the sensing optical fibre is operatively connected to the first channel by means of the sensing chamber.

This means that fluid passing through the first channel and over the sensing element may be sensed by the sensing optical fibre.

The at least part of the sensing optical fibre may be exposed within the sensing chamber. This means that fluid passing through the first channel will also pass over at least a part of the sensing optical fibre.

In embodiments of the invention, the sensor comprises a plurality of sensing optical fibres, each of which sensing optical fibres extends along the sensor such that at least a part of each sensing optical fibre is operatively connected to the first channel by means of the sensing chamber.

In such embodiments of the invention, at least one part of each of the sensing optical fibres may be exposed within the sensing chamber.

By having a plurality of sensing optical fibres, it is possible to optically sense and analyse a plurality of different variables at the same time in order to obtain a complete analysis relative to the diagnosis in question.

In embodiments of the invention the sensing element comprises an electrical sensor extending along the first, fibre such that at least a part of the electrical sensor is operatively connected to the first channel by means of the sensing chamber.

The electrical sensor maybe in the form of an electrical conductor.

In embodiments of the invention where the electrical sensor comprises an electrical conductor, the electrical conductor may be in the form of wire. The wire may be contained within a sensing optical fibre or may be separate to any sensing optical fibres.

In embodiments of the invention the sensing element comprises a sensing optical fibre and an electrical sensor. In some embodiments of the invention, the sensing element comprises a plurality of sensing optical fibres and/or a plurality of electrical sensors.

In embodiments of the invention, the sensor further comprises a second channel extending along, the sensor, which second channel is operatively connected to the first channel.

The second channel may be used as a cleaning channel and may therefore be used as a means for enabling a cleaning fluid to be passed through the sensor when the sensor is not in use. In other embodiments of the invention, the second channel may have a different purpose. In some embodiments of the invention, the first and second channels are both used to pass a fluid to be analysed through the sensor.

In embodiments of the invention comprising a first channel and a second channel, when the sensor is in a sensing mode, fluid from the ambient surroundings to be analysed may be pulled or drawn through both the first channel and the second channel after entering the sensor via the inlet.

In such embodiments of the invention, when the sensor is in a cleaning mode, a cleaning fluid may be flushed through the first channel via the outlet of the sensor and may pass through both the first channel and the second channel before emerging also through the outlet of the sensor.

The sensor may comprise a connector which connects the first channel to the second channel. The connector may be in the form of a connecting channel.

In embodiments of the invention comprising a second channel, the sensor may further comprise a second pump operatively connected to the second channel. In such embodiments of the invention, the first pump may be operatively connected to the first channel.

In embodiments of the invention comprising a second pump, the second pump may comprise a syringe pump or a reservoir pump of the type described here and above with reference to the first pump.

In such embodiments of the invention, the first and second pumps work together to create an appropriate flow of fluid through the sensor depending on, for example whether the sensor is in an operative mode, or a cleaning mode.

In other embodiments of the invention, the first pump is operatively connected to both the first and second channels and serves to pump, fluid through both the first and second channels as required. In such embodiments a single pump only is required.

In embodiments of the invention, the sensor comprises a third, channel operatively connected to the first channel by means of the sensing chamber.

The third channel may be used in any convenient way, and in embodiments of the invention, the third channel is used to enable a reagent to be mixed with the fluid that is to be analysed by the sensor.

In embodiments of the invention, the sensor element comprises a first probe element removably positionable within the first channel.

In such embodiments of the invention, the sensing element is formed separately from and is removable from the first fibre.

This can be useful if, for example it is required to use different types of sensing elements. A first probe element may then be readily removed and replaced with a different first probe element in order to sense a different variable.

The first probe element may be formed from any suitable material and may for example be a drawn fibre.

In such embodiments of the invention, the first probe element is shaped such that when positioned within the first channel, voids are formed between the first channel and the probe element. In such embodiments of the invention, the microfluidic pattern forming part of the microfluidic flow channel is formed from the voids formed between the first probe element and the first channel.

In embodiments of the invention, the sensor comprises a second probe element adapted to be removably positionable within the second channel.

In embodiments of the invention the sensor comprises a light sensitive material operatively connected to one or more of the first, second and third channels. The light sensitive material may be light actuated, or may be any other material that expands when exposed to light.

In embodiments of the invention, the light sensitive material is, patterned with a microfluidic pattern.

In some embodiments of the invention the sensor comprises a switch adapted to switch the sensor between a sensing configuration and a clearing configuration. In embodiments of the invention in which the microfluidic flow channel comprises a light sensitive material, the switch may comprise a first switching optical fibre and a second switching optical fibre, the first switching optical fibre being operatively connectable to a cleaning channel and the sensing element, and the second switching optical fibre being operatively connected to a drain channel and the sensing element.

In such embodiments of the invention, the sensor may comprise an end portion at an end of the sensor, which end portion is formed from the light sensitive material.

The light sensitive material may be a light actuated material, such as a thermal actuated polymer of the type described in International patent application No. WO 2012/142235 and WO 2017/120594. Alternatively the light sensitive material may be any other material that is adapted to expand when exposed to light.

The light sensitive material is adapted to fit over an exposed end of the sensor and thus has dimensions that are similar to the cross-sectional dimensions of the sensor.

The end portion may be patterned with a microfluidic circuit. The microfluidic circuit may be made by moulding polymer during its curing or ablation with lasers, FIB or classical milling.

In use, in order to switch the sensor into the sensing configuration, light may be shone on the first switching optical fibre. This causes the light sensitive material to expand, thereby blocking a flow path from a sensing element to the cleaning channel. When it is required to switch the sensor in the cleaning configuration, light is shone on the second switching optical fibre. This causes the light sensitive material to expand over the second switching optical fibre, thus blocking the flow path between a sensing element and the drain channel.

In embodiments of the invention the sensor further comprises a data analysis unit connectable to a proximal end, of the first fibre, wherein the sensing optical fibre operatively connects the sensing element to the data analysis unit.

The invention will now be further described by way of example only with reference to the accompanying drawings in which;

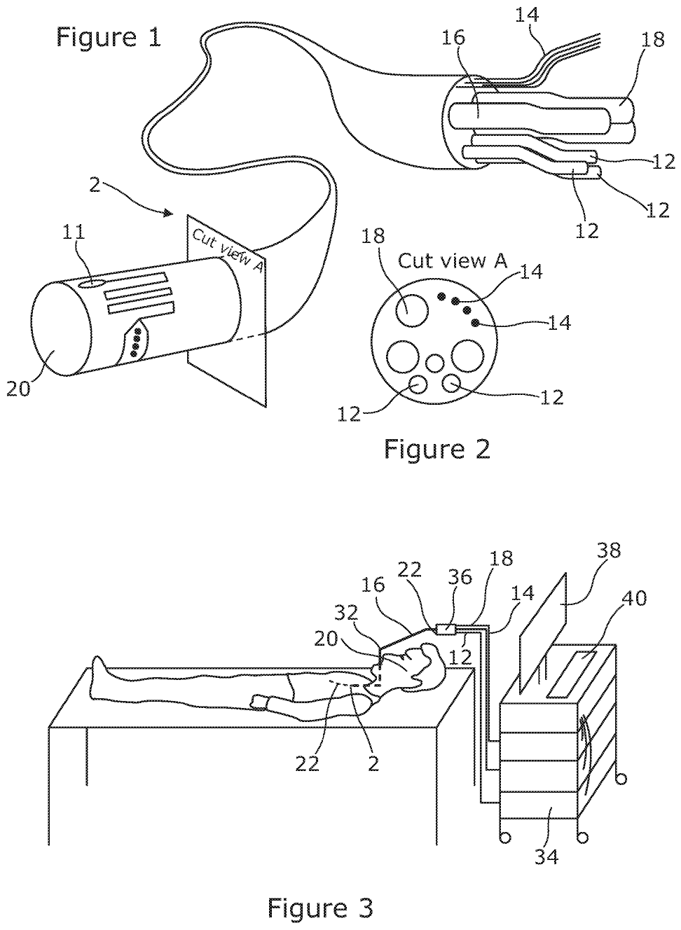

FIG. 1 is a schematic representation of a sensor according to a first embodiment of the invention;

FIG. 2 is a cross sectional representation of the sensor of FIG. 1;

FIG. 3 is a schematic representation of a diagnostic system incorporating a sensor according to embodiments of the invention;

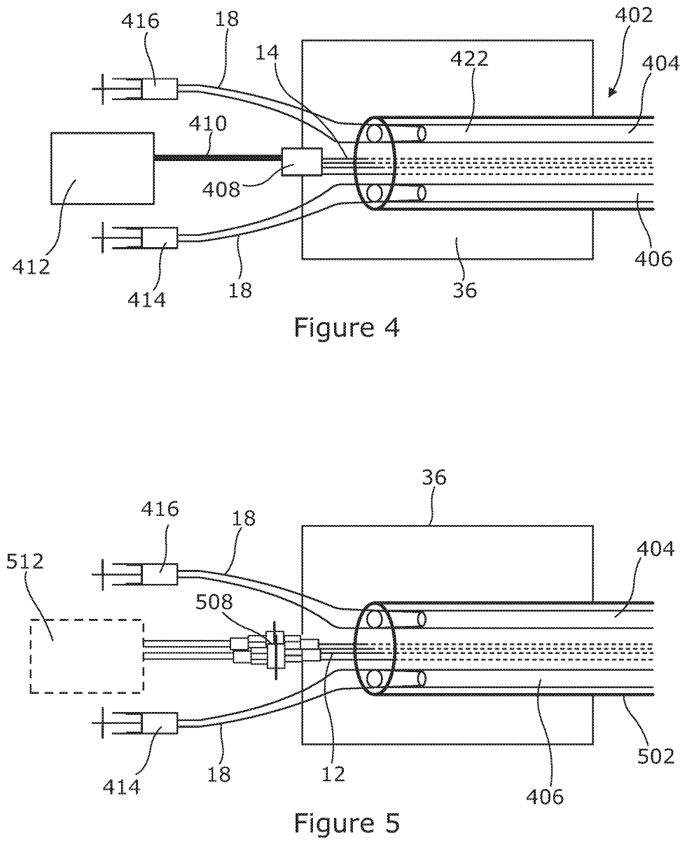

FIG. 4 is a schematic representation of the proximal end of a sensor according to embodiments of the invention;

FIG. 5 is a schematic representation of the proximal end of another embodiment of a sensor according to the invention;

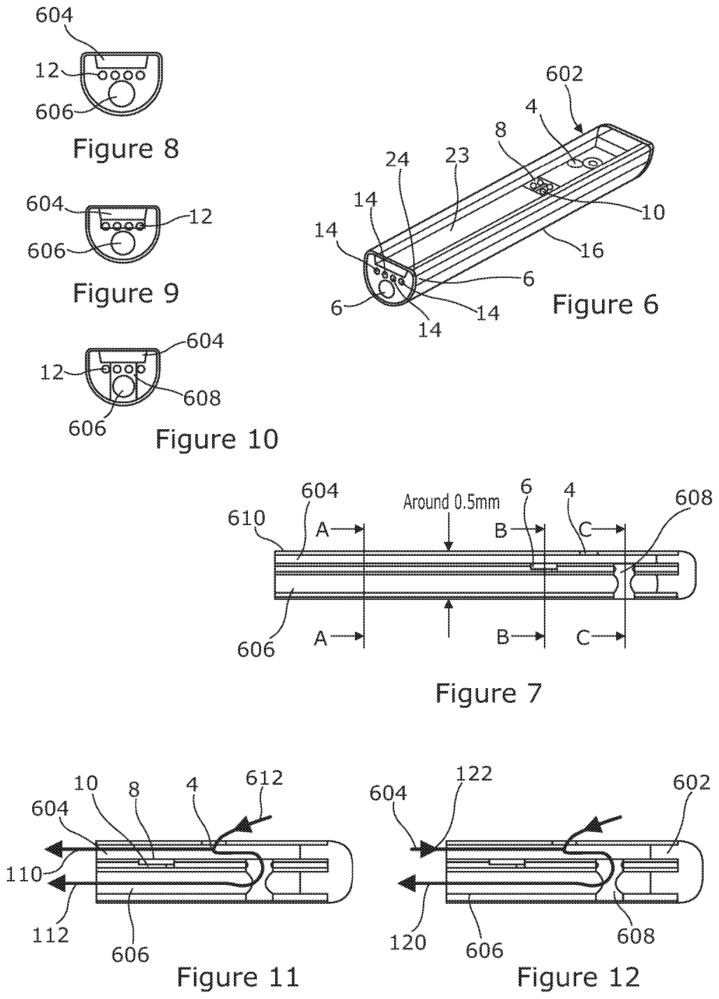

FIG. 6 is a schematic representation a sensor according to another embodiment of the invention;

FIG. 7 is a cross sectional representation of the sensor of FIG. 6:

FIGS. 8, 9 and 10 are cross sectional representations taken along A-A, B-B and C-C respectively as shown in FIG. 7;

FIG. 11 is a schematic representation of part of the sensor of FIG. 6 showing the sensor in a sensing configuration;

FIG. 12 is a schematic representation of part of the sensor in FIG. 6 shown in a cleaning configuration;

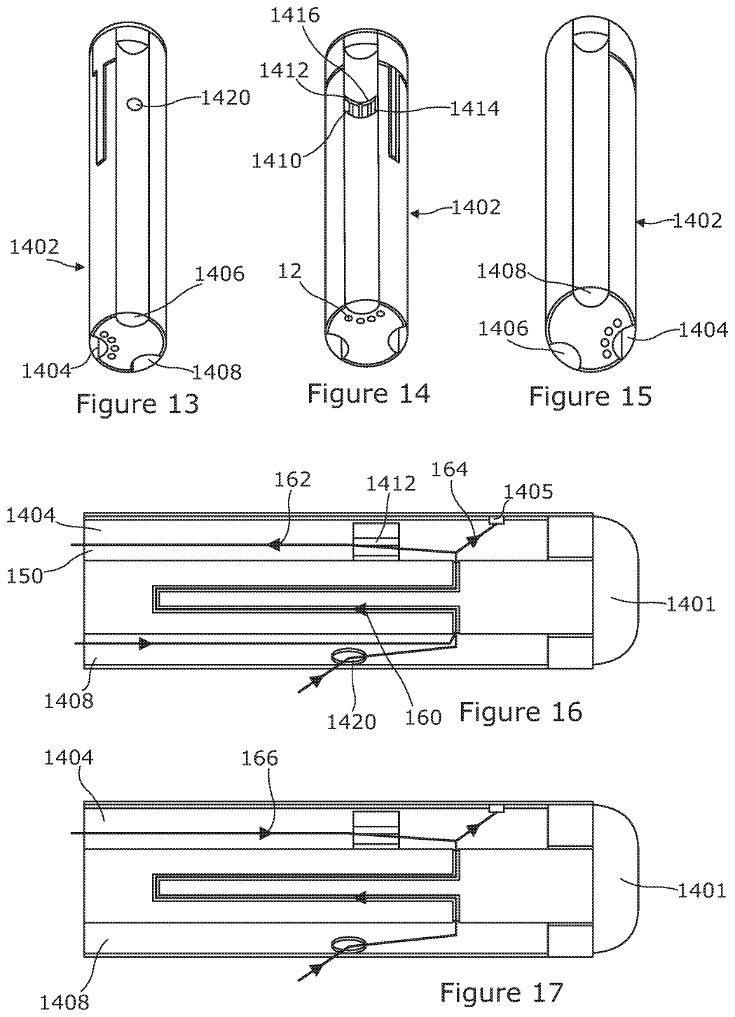

FIGS. 13 to 15 are schematic representations of a sensor according to another embodiment of the invention having three channels;

FIGS. 16 and 17 are schematic representations of the sensor shown in FIGS. 13 to 15 in a sensing configuration and a cleaning configuration respectfully;

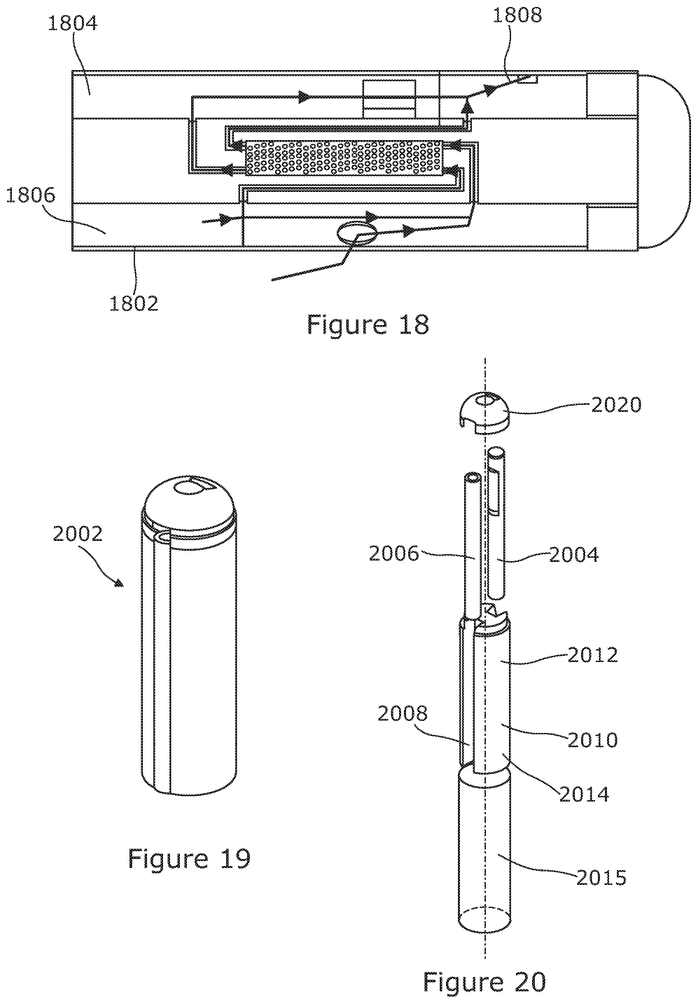

FIG. 18 is a schematic representation of a portion of a sensor of the type shown FIGS. 13 to 15 and comprising a microfluidic mixture;

FIG. 19 is a schematic representation of a sensor cording to another embodiment of the invention comprising two channels;

FIG. 20 is an exploded perspective view of the sensor of FIG. 19;

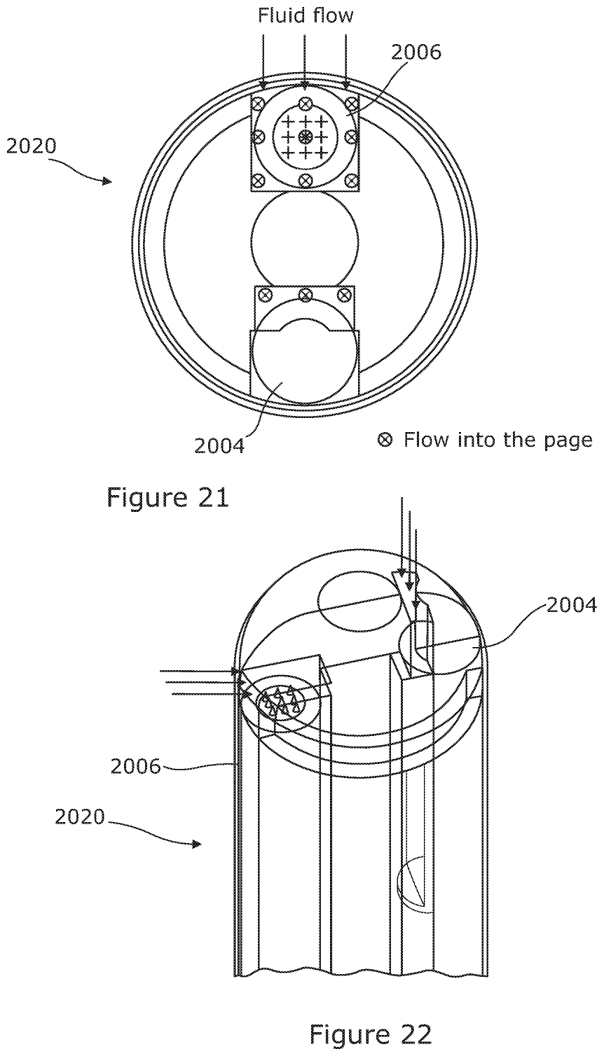

FIG. 21 is a top view of the sensor of FIG. 19;

FIG. 22 is a perspective view of the tip of the sensor of FIG. 20 showing fluid inlets forming part of the sensor;

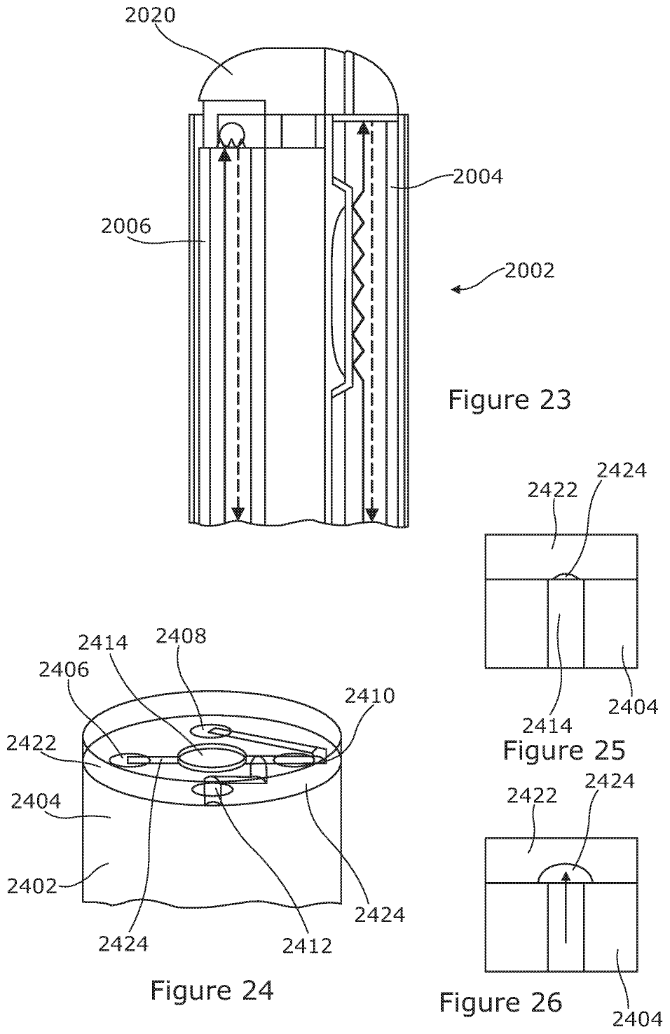

FIG. 23 is a schematic representation of the sensor of FIG. 19 showing the sensing regions;

FIG. 24 is a detailed perspective view of the tip of a sensor according to another embodiment of the invention and having an optically controlled valve;

FIG. 25 is a schematic representation of the valve forming part of the sensor tip shown in FIG. 24 in an open position;

FIG. 26 is a schematic representation of the valve of FIG. 25 in a closed position;

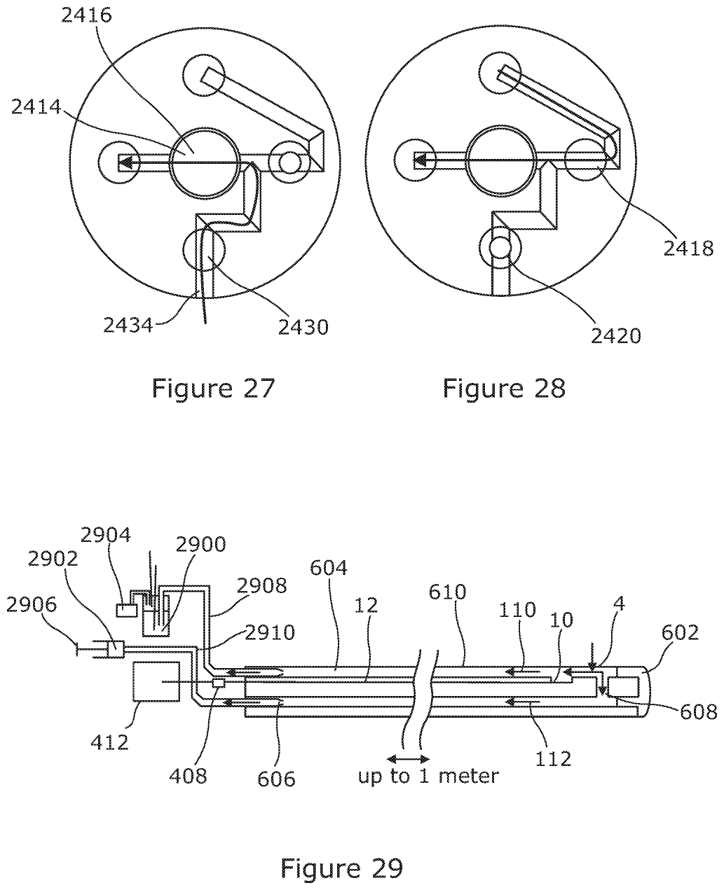

FIGS. 27 and 28 are schematic views from above of the sensor of FIG. 28 in the sensing configuration cleaning configuration respectively; and

FIG. 29 is schematic representation showing flow of fluid through the embodiment of the invention shown in FIG. 11.

Referring first to FIGS. 1 and 6 to 12 a sensor according to an embodiment of the invention is designated generally by the reference numeral 2. The sensor comprises a microfluidic sensor comprising a distal end 20 and a proximal end 22 and an inlet 4 and an outlet 6 as shown particularly in FIG. 7. The sensor 2 also comprises a sensing chamber 8 positioned between the inlet 4 and the outlet 6, and a sensing element 10. In this embodiment of the invention, the sensing element comprises a plurality of sensing optical fibres 12 and electrical wires 14, each of which is partially exposed within the sensing chamber 8.

The sensor 2 comprises a fibre 16 formed from a drawable material. In order to form the sensor 2, the fibre 16 is drawn in the shape shown specifically in FIG. 6 from a preform.

The optical sensors 12 and/or electrical wires 14 are placed inside the fibre, either by co-feeding during the drawing process, or by sliding in after the drawing process.

Once the electrical wires and/or optical sensors have been placed inside the sensor 2, the wires and/or optical sensors may be exposed by removing some of the fibre material to expose the sensing chamber.

The microfluidic patterning may be achieve using any convenient method such as laser patterning, FIB patterning, moulding, micro-milling. Other methods may also be appropriate.

The sensor 2 is adapted to sense variables either electrically or optically by means of the sensing optical fibres 12 and the electrical wires 14. Sensing is achieved by the functionalisation of a surface of the sensor 2.

The sensing elements may be positioned anywhere along the length of the sensor 2. For example, the sensing chamber may be positioned anywhere along the length of the sensor 2 and/or at a distal end 20 of the sensor 2. By means of the present invention therefore it has been possible to design a microfluidic chip that is connected to both a measurement unit and fluid input all on a single fibre 16. The input of the sensor 2 allows liquid sampling to take place in vivo. The sensing is incorporated inside the sensing chamber 8 by a functionalisation of the surface of the sensor 2 which is in the form of a microfluidic chip.

The sensor 2 is connected at the proximal end 22 to a tunable pressure reservoir 2900 (shown in FIG. 29) in order to generate a constant positive or negative pressure.

By means of the sensing optical fibres 12 and the electrodes 14, measurements, may be made electrically and/or optically.

By means of the present invention therefore the sensor 2 may be used for in vivo chemical sensing and a distal end of the fibre 16 may be positioned at a point where measurements are to be taken.

Because all the components of the sensor 2 are within a single fibre 16, a controlled laboratory-like environment is achieved for the sensing process. This helps to reduce or eliminate noise and other interactions that may adversely affect open sensors. In addition, the sensing chamber 8 is protected from mechanical damage during insertion and throughout the measurement process.

By means of the present invention, all connections are provided through a single fibre and thus the sensor 2 is compact and robust.

The sensing environment passing through the sensing chamber is constant and known thus allowing repeatable and quantifiable measurements.

The invention may comprise a multi-material fibre 16 having inherent electrical, optical and fluidic channels formed therein with desired geometries.

The sensor 2 may be made using any convenient methods such as focussed ion beam (FIB), laser patterning, drilling, and milling. Such processes may be used to add non-axial features to the fibre allowing channel connection or complex microfluidic features to be incorporated into the sensor 2. The sensor 2 further comprises fluid tubes 18 for allowing fluid to pass through the sensor 2.

A sensor 2 according to embodiments of the invention has a wide range of applications but is of particular use within the medical field.

Referring to FIG. 3, a medical diagnostic system 30 comprising a sensor 2 of the type shown in FIG. 1 is illustrated schematically. In the illustrated embodiment, a catheter 32 serves to connect the sensor 2 to an analyser 34. The catheter 32 is connected to an interface box 36, and the sensor 2 extends through the catheter to the fibre interface box 36. The optical fibres 12, electric wires 14 and fluidic tubes 18 pass through the fibre interface box 36 to the analyser 34. The analyser 34 comprises a user interface 38, 40 which may be in the form of a monitor 38 and keyboard 40 for example.

The size of components such as the user interface 38, 40 and, the analyser 34 may vary to suit the application to which the sensor 2 is being put.

In the illustrated embodiment, the sensor 2 has been inserted into the lungs of a patient via the mouth of a patient. However, a sensor according to embodiments of the invention may be adapted to be inserted through other orifices, natural or otherwise, in order to take measurements in an appropriate part of the body.

As may be seen particularly from FIG. 3, the proximal end 22 of the fibre 16 is connected to the fibre interface box 36, and the distal end 20 of the fibre 16 is positioned appropriately within the body of the patient.

Referring now to FIGS. 4 and 5 the proximal end of a sensor accords to two embodiments of the invention is shown in more detail in order to illustrate how a sensor 2 according to embodiments of the invention may be connected to the fibre interface box 36.

Referring first to FIG. 4, the proximal end 422 of a sensor 402 according to embodiments of the invention is illustrated. In this embodiment of the invention, the sensor 402 comprises a first channel 404 and a second channel 406. The channels 404, 406 will be described in more detail below.

The fibre further comprises electrical wires 14 which are connected via an electrical connector 408 and a connection cable 410 to an electrical measurement station 412.

Fluidic tubes 16 are connected to syringe pumps 414 and 416 respectively.

Turning now to FIG. 5, a sensor 502 according to another embodiment of the invention is illustrated. Parts of the sensor 502 which are similar to those of the sensor 402 have been given corresponding reference numerals for ease of reference.

In this embodiment of the invention the sensor 502 comprises optical fibres 12 which are connected via optical fibres connectors 508 to an optical spectroscopy set up 512.

In some embodiments of the invention both electrical wires (or electrodes) 14 and optical fibres 12 will be present in a sensor according to embodiments of the invention. Such a sensor may comprise a multi-material fibre having inherent electrical, optical and fluidic channels formed therein with desired geometries.

Referring now to FIGS. 6 to 12 and 29 an embodiment of the invention in the form a sensor 602 is illustrated. In this embodiment of the invention the sensor 602 comprises inlet 4 and two outlets 6. Positioned between the inlet 4 and the outlets 6 is a sensing chamber 8 with a sensing element 10 exposed therein. The sensing element 10 comprises portions of electrical wires 14.

The sensor 602 also comprises, a first channel 604 which forms a microfluidic channel and a second channel 606. The first and second channels 604, 606 are connected by a connecting channel 608. The sensor 602 is sealed by a cover 23 formed from heat shrink polymer forming a seal 610. This allows easy machining of the surface of the sensor 602 using techniques such as focus ion beam or laser patterning.

The first channel 604 is operatively connected to the inlet 4 and to first outlet 6. The second channel 606 is operatively connected to the first channel 604 by means of the connecting channel 608 and is also connected to the second outlet 6. One or more pumps of the type shown in FIGS. 4 and 5 are operatively connected to each of the channels 604, 606.

In order the functionalise the electrodes 14, a surface of each of the electrodes is cleaned electrochemically with 50 mM sulphuric acid. The electrodes are then dried and a layer of conductive platinum nanoparticles is deposited on the electrodes to increase the surface area of the electrodes.

Next, an ion-sensitive cocktail containing ionic sites such as nitrophenyl octyl ether, ionophore specific for the specific analyte of interest such as pH ionophore, sodium, potassium, calcium ionophores, etc.

In the case of enzyme sensing, a different cocktail is drop casted with an enzyme that sensitive towards the analyte of interest which is crosslinked to bovine serum albumin using glutaraldehyde. Several layers of biocompatible membrane layers such as polyurethane are deposited at the end to achieve protection and long life-time response.

In order to ensure a good seal, the cover 23 may be made from a heat shrinkable polymer. In such embodiments of the invention, the sensor 2 is first of all fit with a loose fitting heat shrinkable cover. The sensor is then heated so that the cover shrinks tightly around the fibre.

In other embodiments, the step of functionalisation may be carried out after the sensor 2 has been sealed.

In order to take a measurement, fluid from the surrounding environment is caused to enter the sensor 602 via inlet 4 and is drawn through the sensing chamber 8 over the sensing elements 10 by means of the one or more pumps (shown in more detail in FIG. 29). In the sensing configuration, as shown in FIG. 11, fluid to be sampled is caused to flow along both the first and second channels 604, 606 as shown by the arrows 110, 112 in FIG. 11.

In this embodiment of the invention, the first channel 602 is connected at its proximal end to a tuneable pressure reservoir 2900 (FIG. 29) in order to generate a constant positive or negative pressure.

In the sensing configuration, the electrodes are used to carry out electrochemical measurements in order to analyse the liquid being drawn through the sensor 602.

In the sensing configuration, as shown in FIG. 11, the one or more pumps create suction through channel 604 and 606 in order to pull, or suck fluid to be analysed from the surrounding environment into the sensor 602 via inlet 4 and through the first and second channels 604, 606 as shown in FIG. 11.

By setting a negative pressure, external liquid will ow inside the channel from the opening 24 and will pass over the electrodes 14.

In contrast, when the sensor 602 is in the cleaning configuration, as shown in FIG. 12, the one or more pumps cause suction through channel 606 and flushing through channel 604 as shown by the arrows 120, 122. A cleaning solution may be pulled into the sensor via inlet 4 and channel 604, and may be drawn through the sensor and out through channel 606. Because of the flow created in this way, there will be no leakage of the cleaning solution into the environment due to the flushing taking place through channel 604 which prevents cleaning fluid from exiting via inlet 4.

In the cleaning mode, the reservoir may be filled with a cleaning solution which will flow through channel 604 and through the opening 24 into the in vivo environment. The cleaning fluid will therefore need to be a biocompatible solution such as a saline solution.

A cleaning solution may be pulled through the sensor by increasing the pressure of the reservoir to a positive pressure.

The flow inside the sensor 602 is shown in more detail in FIG. 29.

The flow with the sensor 602 may be controlled either by syringe, pumps 2902 or by reservoir pumps 2904 which tune the pressure of the reservoir 2900. In some embodiments of the invention there may be a mixture of syringe pumps 2902 and, reservoir pumps 2904.

In this embodiment of the invention each of the channels 604, 606 is connected to either a syringe pump 2902 or a reservoir 2900.

The syringe pump 2902 comprises a plunger 2906, the movement of which is controlled by a linear motor (not shown). The linear motor provides a constant speed to the plunger which in turn causes a constant flow of fluid, outside the syringe pump 2902. The flow may be either positive or negative depending on whether the plunger 2906 is pushed or pulled by the linear motor.

The reservoir pump 2904 controls the pressure of air inside the reservoir 2900 which is sealed. The pressure acts on fluid within the reservoir 2900 to cause a flow of fluid. If a positive pressure is applied by the pump 2904, fluid will flow out of the reservoir 2900 towards the sensor 602.

If a negative pressure is applied by the reservoir pump 2904, fluid II flog from the sensor 602 to the reservoir 2900.

The reservoir 2900 and the syringe pump 2902 are connected to the sensor 602 by means of fluid tubes 2908 and 2910 respectively. The tubes 2908, 2910 may be made from any suitable material such as polymer or glass although other materials may also be suitable. The tubes 2908, 2910 are connected to the channels 604, 606 respectively, A seal is formed between a respective fluid tube 2908, 2910, and channel to 604, 606 in order to ensure efficient flow of fluid between the reservoir 2900, syringe pump 2902 and the sensor 602.

In the embodiment shown in FIG. 29, fluid enters the sensor 602 via inlet 4 and then flows via channels 604, 606 towards the reservoir 2904 and syringe pump 2902.

The sensor 602 may be used as an electrochemical sensing mechanism.

Electrochemical detection of different targets such as electrolytes and biomolecules can be realised onto one platform by using the sensor 602. The microfluidics forming part of the sensor 602 ensure a better control of the fluids at the surface of the electrodes.

The principle of work involves a setup where the potential or indicator electrode is measured against a reference electrode under zero-current conditions. Solid-state ion selective electrodes are based on low soluble salts of the ion of interest. Changes of the transmembrane potential is proportional to the analyte concentration.

In the case of biomolecule detection, the indicator electrode has an enzymatic layer and outer protective layer limiting our working range. Detection of the changes in the current output is proportional to the concentration of the analyte of interest.

Another important method of electrochemical detection is through the immobilisation of an antibody onto the electrode surface which has an effect on the amount of the immobilised protein and in current signal of the protein. Microfluidic-based electrochemical sensing is a very sensitive, rapid and specific way of detection. Changes in the current output with time is proportional to the analyte concentration. In one embodiment of the invention the sensor may be used as an affinity biosensor which comprises a biological recognition element such as an antibody, receptor protein, biomimetic material, or DNA interfaced to a signal transducer, where the measured signal is related to the concentration of an analyte. The electrochemical detection offers a less expensive means of reading the signal. If the electrochemical reporters and the electrolyte are chosen correctly, the electrical signal is stable over e and may have less interferences compared with optical detection.

Referring now to FIGS. 13 to 18 another embodiment of the invention is illustrated. This embodiment comprises a sensor 1402 comprising three channels 1404, 1406 and 1408.

The sensor 1402 is formed using a similar method to that described hereinabove with reference to the embodiment shown in FIGS. 6 to 12. Each of the channels 1404, 1406 and 1408 is sealed by means of a heat shrink polymer. The sensor 1402 further comprises a sensor tip portion 1401 which is sealed by any convenient means for example by applying a liquid polymer drop at the tip 1401. The polymer Will naturally fit the microfluidic channels by capillary forces depending on choices of polymers, it may be cured, (solidified) with time, heat or UV exposure.

Once the heat shrink polymer and the polymer at the tip of the sensor have been applied, the heat shrink polymer is pierced to form an aperture 1420. This allows an input from the external environment to the microfluidic.

A first channel 1404 has formed therein a microfluidic flow structure 1410 and a sensing chamber 1412 containing a sensing element 1414 extending therethrough. The channel 1404 is similar to the channel 604 described hereinabove with reference to FIGS. 6 to 12. The sensor 1402 comprises a plurality of sensing optical fibres 1412 portions of which are exposed within the sensing chamber 1412 to act as a sensing element 1414. The sensing chamber 1412 may also comprise electrodes 1416 in order that electrical conductors may also be used to form part of the sensing element 1414.

Each of the channels 1404, 1406, 1408 are operably connected to one another via connector 1405. Channel 1408 serves as a drain channel and channel 1406 serves as a reagent channel.

The channels 1404 and 1406 are connected to a tuneable pressure reservoir (FIG. 29) in order to generate a constant positive or negative pressure. The connector 1405 is connected to channel 1408, and this channel is also connected to the pressurised reservoir.

The pressurised reservoir connects to channel 1408 such that the channel 1408 is filled with a liquid which mixes with liquid surrounding the sensor 1402. The mixing liquid may be, for example an anticoagulant to prevent blocking of the microfluidic sensor in situations where the sensing environment comprises blood and/or protein which fixes to a particular bioelement and which may be detected by means of electrode 1412.

A negative pressure of the reservoir is set on the input connected to channel 1404 in order to direct the mixed flow to the sensing region 1408 of the sensor 1402.

Electrochemical sensing may then take place by means of the electrodes 1412.

In the sensing configuration as shown in FIG. 16, fluid to be analysed is drawn into the sensor via inlet 1402. The fluid sample drawn in in this way will pass through the sensor on a path indicated by arrows 160, through the mixing area where additives may be mixed with the sample and then along through channel 1404 and through the sensing area 1412 before exiting via an outlet 150 at an end of the channel 1404.

The pathway 160, 162 is relatively long. This ensures that the fluid sample with which the channel is filled is thoroughly mixed. The sensor 1402 ensures a non-turbulent flow of fluid within it, and therefore a long path is required to enable the mixing to take place via diffusion processes.

A more convoluted shape for the pathway 160 could be used in other embodiments in order to improve diffusive mixing processes.

Some of the sample of fluid may also exit via the drainage channel 1406 as indicated by arrow 164.

In the sensing configuration, a reagent or other additive may enter the sensor in 1402 via an inlet at an end of the channel 1408. The reagent may then be pulled through the sensor by means of a pump to mix with the sample to be analysed to pass through the sensor along the same path identified by arrows 160, 162 as described hereinabove with reference to the fluid sample.

Channel 1408 may thus be used to mix for example an additive with the sample before the sample is tested.

In a cleaning configuration as shown in FIG. 17, the sensing channel 1404 may also be used to allow a cleaning solution to pass through the sensor 1402 when the sensor is not being used to measure a sample. In the cleaning configuration cleaning solution will be drawn through the sensor 1402 by means of a pump (not shown) to a pass through the sensor 1402 in the direction of arrow 166.

This may be achieved by setting a negative pressure by means of the tuneable pressure reservoir.

Turning now to FIG. 18, an embodiment of the invention which is suitable for the separation of red blood cells and platelets from white cells and circulating tumour cells is shown. The sensor in this embodiment is designated generally by the reference numeral 1802. This embodiment is similar to the embodiment shown in FIGS. 13 to 17 in that the sensor 1802 comprises three channels 1804, 1806 and 1808. Channel 1804 is a cleaning channel, channel 1806 is a buffer channel.

The sensor 1802 is formed using the method described hereinabove with reference to previous embodiments. Specifically, the sensor 1802 is formed by drawing a fibre containing four electrodes or optical fibres such that these electrodes or optical fibres are positioned beneath one of the channels 1804, 1806, 1808.

Focussed ion beam (FIB) is used to open the fibre to form a window in order to position an electrode at an appropriate position within one of the channels.

The end of the channel may then be sealed with UV curable resist.

The electrodes and/or optical fibres can then be functionalised.

In this embodiment of the invention, the channels are isolated from the external environment through use of a heat shrink polymer.

Referring now to FIGS. 19 to 23, a sensor 2002 according to another embodiment of the invention is illustrated schematically.

The sensor 2002 is adapted to work with a constant flow of fluid that is to be analysed and is able to continuously sense the fluid passing through the sensor 2002.

The sensor 2002 comprises a side sensing optical fibre 2004, and a tip sensing optical fibre 2006. The tip sensing optical fibre 2006 is formed from a multimode optical fibre cut to an appropriate length. The length may be between 10 and 15 cm for use in shallow regions such as the oral cavity, nasal cavity, brain, open incision etc., and between 30 cm to 1 m for use in deep area such as the lungs, intestines, liver, stomach etc.

The polymer protective jacket surrounding the fibre may then be removed using a fibre stripper. Next the end tips of the optical fibre may be cleave using a fibre cleaver.

The fibre is then placed and clamped within a 3D printed fibre holder. Direct laser writing, or two photon polymerisation (2PP) of photo resist of microstructures on the tip of the fibre with femtosecond near infrared laser takes place. After that step, the development of the polymerised microstructures by emersion in a developer such as propylene glycol methyl ether acetate takes place.

Next, the microstructure is metallised with a thin layer of approximately 100 nm of a noble metal such as gold or silver by metal deposition techniques. Suitable techniques include electron beam deposition, thermal evaporation, sputtering etc.

After these steps have been carried out, the fibre is ready to act as the sensor 2006 shown in FIG. 20.

The side sensing optical fibre 2004 is formed using similar steps, up to the point where the fibre may be placed and clamped within a 3D printer fibre holder.

At this point, a length of the cladding, of the optical fibre having a length approximately 1 to 2 cm is removed to expose the core of the fibre. The cladding may be removed all around the core, or just in one direction. The cladding may be removed using any suitable technique such as chemical etching using ammonium fluoride or hydrofluoric acid, etc, or by mechanical polishing or milling techniques.

A section of the exposed core is then coated with a layer of a metal or with multiple layers of different metals including, but not limited to: gold; silver; platinum and copper.

If gold is used, an initial layer of chromium of approximately 5 nm thickness may be deposited to the core of the fibre before the gold is deposited in order to facilitate proper adhesion of the gold layer to the fibre.

By following these steps the sensor 2004 is formed.

In this embodiment of the invention, the tip sensing optical fibre 2006 is used to carry out Surface Enhanced Raman Spectroscopy sensing (SERS) whilst the side sensing optical fibre 2004 is adapted to perform Surface Plasmon Resonance (SPR) sensing.

SPR optical sensing is based on the change of local refractive index. This means that fibres adapted to sense this way must be chemically functionalised in order to detect an particular element.

SERS sensing on the other hand measures a characteristic spectrum that can be matched to a database to identify a particular element. This means that SERS, can be used without any functionalisation. However, functionalisation can still be performed to increase the sensitivity of the measurement to a particular element such as bacteria, a cell or protein.

In order to functionalise the fibres the topmost metal layer of the sensing region is coated with chemical, biochemical or nanoparticle moeities that are intended to sense the analyte of interest by exposing the metal coated region to a solution/suspension of these materials. The moieties for chemical sensing could include, but are not limited to: crown ethers, calixarenes, other synthetic ionophores, dyes, etc. Biochemical moieties for biological sensing could include, but are not limited to, antibodies, antigens, proteins, biological ionophores, etc. The nanoparticles would facilitate LSPR (localised surface plasmon resonance) sensing and these nanoparticles could be gold, silver, etc. Furthermore, these nanoparticles would then be functionalised with chemical or biochemical moieties for sensing. Solutions/suspensions of chemicals and/or biochemicals to enable sensing will be used to functionalise the metal coated area's of the optical fibres using standard chemical techniques. Then cleaning/washing steps with solvents or water or biological buffer solutions will be achieved using standard chemical techniques, to ensure the functionalised sensing region/nanoparticle region is prepared for analyte sensing.

This functionalisation step can be made before the assembly in the main fibre but also after step 6 below by using the microfluidic connection of the fibre to provide the coating solution as well as the rinsing solution.

The sensor 2002 may be assembled using the steps set out below:
1. Drawing a macro-channel supporting fibre (2010)
2. Sitting the sensor fibres in the channels (2008). The channels are designed larger than the fibre so a spacing remain allowing a microfluidic canal between the channel and the fibre
3. Insertion of the assembly of 2004, 2006, and 2010 in the heat shrink tubing 2015
4. Closing the head of the assembly with a micro-machined or 3D-printed cap 2020, which has desired openings for fluid delivery
5. Heat shrink the heat shrink tubing 2015
6. This assembly becomes the component 2002 in FIG. 19

Together the fibres 2004 and 2006 form part of the sensing element of sensor 2002. The optical fibres 2004 and 2006 are shaped to fit within channels 2008 formed in body portion 2010 of the sensor 2002.

The shape of the fibres 2004 and 2006 relative to the shape of the channels 2008 is such that when the fibres 2004 and 2006 are positioned within the channels 2008, microfluidic channel is formed within each of the channels 2008 by the gaps existing between the fibres 2004, 2006 and a respective channel 2008. The channels 2008 are formed within a fibre 2012 in which microfluidic channel grooves are formed. These microfluidic channel grooves form the sensing chamber in the sensor 2002.

In the illustrated embodiment, when the channel 2008 is designed such that it becomes deeper towards a bottom end 2014 of the body portion 2010. This results in the fibre 2006 being held away from the groove wall forming the microfluidic channel.

For the tip sensing optical fibre 2006, a sensing chamber is formed as will be described hereinbelow.

In this embodiment of the invention, the sensor further comprises a cap 220 engageable with each of the fibres 2004, 2006. Fluid inlets for both fibres 2004, 2006 are formed on the cap 2020. Axial and side openings are designed on the fibres in order to ensure that the flow of fluid from outside of the sensor passes over the sensing region. The diameter of the axial and side openings may be adjusted in order to ensure an appropriate intake flow rate. The smaller opening will result in a smaller flow rate.

A constant flow of fluid is achieved through use of syringe or pressure pump at a proximal end of the sensor. The constant flow is achieved by creating a negative pressure through the sensor 2002. This results in a constant flow of fluid flowing from the surrounding environment through inlets passing into the tip sensing region and a side sensing region respectively.

Referring now to FIGS. 24 to 28, a sensor 2402 according to another embodiment of the invention is illustrated.

The sensor 2402 is formed from a polymer fibre 2404 and is formed with channels (in this case 5 channels) 2406, 2408, 2410, 2412 and 2414. A fibre 2416 is placed within channel 2414. In this embodiment of the invention, the fibre is prepared to sense with the tip and is therefore similar to the probe 2006 shown in FIGS. 19 to 23 and described above. Fibres 2418 and 2420 are fitted into channels 2410 and 2412 respectively.

In order to maintain the fibres in place in the respective channels, as well as to prevent any leakage, glue is used to fill any spaces between a respective fibre and the channel in which it is held. An end portion 2422 is formed from a light actuated material such as a thermal actuated polymer or any other material that expands when it is exposed to light. The end portion 2422 is cut to fit over an end face 2424 of the sensor 2402.

The end portion 2422 is patterned with a microfluidic pattern which is similar to the microfluidic patterns described hereinabove with respect to the previous embodiments. This results in a microfluidic channel 2424 being formed.

In this embodiment, the microfluidic pattern is formed by moulding the polymer forming the end portion 2422 during curing or by ablation with lasers, FIB or classical milling.

The end portion 2422 may be fitted to the sensor 2402 by any convenient means such as by using an adhesive.

The channel 2406 is connected to a depression pump (not shown) to constantly generate a vacuum force in order to attract liquid to be tested into the sensor 2402. The channel 2408 is connected to a cleaning liquid at ambient pressure.

Referring now to FIG. 25 no light is shone through fibre 2420 which is fitted into channel 2412. This means that the polymer end portion 2422 maintains the shape shown in FIG. 25. This in turn means that liquid may pass through channel 2414.

Turning now to FIG. 26, light is shone through optical fibre 2420 in channel 2412. This results in the expansion of the polymer forming the end portion 2422 in a region close to the tip 2424 of the fibre 2416. This expansion results in the polymer forming the end portion 2422 filling the channel space and thereby blocking the channel 2414. In this situation no fluid can pass through the channel 2414.

During sensing, light directed via the fibre 2420 is switched off. This creates a channel between the outside of the microfluidic chip 2430 and end portion 2422. This allows liquid surrounding the sensor 2402 to enter the sensor via an opening 2434 and to then pass through the sensing region created in the end portion 2422 of the sensor 2402 as shown in FIG. 27.

During the cleaning step, light is initially directed onto fibre 2420 to close the connection between the sensing region and the ambient surroundings.

At this point light directed onto fibre 2418 is stopped in order to open the access between channel 2406 which acts as a drain channel, and channel 2408. This causes cleaning liquid to be pulled into channel 2406 then to pass over the sensing region as shown in FIG. 28.

When the sensing process is completed, light is then directed onto fibre 2418 in order to stop the flow of the cleaning liquid.

1. A sensor comprising an inlet and an outlet, a sensing chamber positioned between the inlet and the outlet, and a sensing element operatively connected to the sensing chamber, wherein the sensor comprises a first fibre formed from a drawable material, the fibre comprising a first channel extending between the inlet and the outlet, the sensing chamber being formed within the channel.

2. A sensor according to claim 1, further comprising a seal, sealingly connected to the first fibre.

3. A sensor according to claim 1, wherein the first channel comprises a microfluidic flow channel.

4. A sensor according to claim 3, wherein the first channel comprises patterns etched into the wall of the first channel in the sensing chamber.

5. A sensor according to claim 1 further comprising a first pump operatively connected to the outlet.

6. A sensor according to claim 5, wherein the first pump comprises a syringe pump.

7. A sensor according to claim 5, wherein the first pump comprises a reservoir pump, operatively connected to a reservoir holding fluid.

8. A sensor according to claim 1 wherein the sensing element comprises an optical sensor comprising a sensing optical fibre extending along the sensor such that at least a part of the sensing optical fibre is operatively connected to the first channel by means of the sensing chamber.

9. A sensor according to claim 1 comprising a plurality of sensing optical fibres, each of which sensing optical fibres extends along the sensor such that at least a part of each sensing optical fibre is operatively connected to the first channel by means of the sensing chamber.

10. A sensor according to claim 1 comprising an electrical sensor extending along the first fibre such that at least a part of the electrical sensor is operatively connected to the first channel by means of the sensing chamber.

11. A sensor according to claim 1 further comprising a second channel extending along the sensor, which second channel is operatively connected to the first channel.

12. A sensor according to claim 11 further comprising a second pump operatively connected to the second channel.

13. A sensor according to claim 12, wherein the first pump is operatively connected to the first channel.

14. A sensor according to claim 1 further comprises a third channel, operatively connected to the first channel by means of the sensing chamber.

15. A sensor according to claim 1 further wherein the sensing element comprises a first probe element removably positioned within the first channel.

16. A sensor according to claim 1, comprising a second drawn fibre adapted to be removably positionable within the second channel.

17. A sensor according to claim 1, wherein the sensor comprises a light sensitive material operatively connected to one or more of the first, second and third channels.

18. A sensor according to claim 17, wherein the light sensitive material is patterned with a micro-fluidic pattern.

19. A sensor according to claim 1, comprising a switch adapted to switch the sensor between a sensing configuration and a cleaning configuration.

20. A sensor according to claim 17, comprising a switch comprising a first switching optical fibre and a second switching optical fibre, the first switching optical fibre being operatively connectable to a cleaning channel and the sensing element, and the second switching optical fibre being operatively connected to a drain channel and a sensing element.

21. A sensor according to claim 17, wherein the sensor comprises an end portion formed from the light sensitive material.

22. A sensor according to claim 1 comprising a data analysis unit connected to the proximal end of the first fibre; wherein the sensing optical fibre operatively connects the sensing element to the data analysis unit.
